(19) United States
(12) Patent Application Publication
(10) Pub. No.: US 2018/0249826 A1
(43) Pub. Date: Sep. 6, 2018

(54) BATHROOM MANAGEMENT APPARATUS

(71) Applicant: LG ELECTRONICS INC., Seoul (KR)

(72) Inventors: Jeongyun KIM, Seoul (KR); Daeyun Park, Seoul (KR); Jinhyeon Jeon, Seoul (KR)

(21) Appl. No.: 15/911,360

(22) Filed: Mar. 5, 2018

(30) Foreign Application Priority Data
Mar. 3, 2017 (KR) 10-2017-0027838

(51) Int. Cl. A47B 47/00; A47B 55/00; A47B 81/00; A47B 95/00; F25D 11/00; A61L 2/10

(52) U.S. Cl. CPC A47B 47/0091; A47B 55/00; A47B 81/00; H02J 7/0042; F25D 11/00; A61L 2/10; A47B 95/008

(57) ABSTRACT

A bathroom management apparatus includes: a cabinet having an open front surface and rear surface; at least one frame body configured to be attached to an inner side of the cabinet to reinforce a stiffness of the cabinet; and at least one back bracket attached to a rear of the at least one frame body and coupled to both side frames of the frame body, wherein a length of the back bracket is determined according to a number of frame bodies.

### CROSS-REFERENCE TO RELATED APPLICATION(S)

This application claims priority under 35 U.S.C. § 119 to Korean Application No. 10-2017-0027838 filed on Mar. 3, 2017, whose entire disclosure(s) is/are hereby incorporated by reference.

### BACKGROUND

### 1. Field

A bathroom management apparatus which is installed at a bathroom to store various bath and toilet appliances including towels and to remove humidity in the bathroom is disclosed herein.

### 2. Background

A bathroom is a place that allows users to wash clothes, wash a face, wash hands, shower, and defecate, for example. Since the bathroom may be the most humid place, mold and bacteria may easily breed which may lead to unpleasant odors in the bathroom.

Some bathrooms may dry and deodorize using a ventilation fan. However, since an operation of the ventilation fan is not suitable or it is insufficient to dry the whole bathroom even while the ventilation fan is operated, pollution may occur due to breeding of mold and bacteria.

Accordingly, it is important to prevent mold and bacteria from forming in the bathroom by removing moisture from a bottom of the bathroom, and by drying wet bath and toilet appliances such as wet towels. Further, various facilities such as a washstand, a toilet, a mirror, a towel rack, and a toothbrush holder, for example, as well as a storage room for storing various bath and toilet appliances including towels may be installed in the bathroom. Meanwhile, a user may use electronic products such as hair dryers and shavers and apply make up in the bathroom.

If a bathroom management apparatus integrating a function of a toothbrush sterilizer, a function of a cosmetics refrigerator, and a charging function of the electronic products based on a function of the storage room and a dry function of the bathroom is developed, the space utility of the bathroom may be increased. Further, if a bathroom management apparatus independently including various function modules including a storage module having a function of the storage room, an air conditioning module having a drying function of the bathroom, a sterilizing module having a function of a toothbrush sterilizer, a refrigerating module having a function of a cosmetics refrigerator, and a charging module having a charging function of the electronic products is developed, the user may select necessary modules from the function modules to include the selected modules in the bath management apparatus.

Since various numbers of the function modules may be provided according to the needs of a user, when the user buys the bath management apparatus, the size of a frame used to install the function module according to the number of the function modules ordered by the user may be adjustable.

The above references are incorporated by reference herein where appropriate for appropriate teachings of additional or alternative details, features and/or technical background.

### BRIEF DESCRIPTION OF THE DRAWINGS

The embodiments will be described in detail with reference to the following drawings in which like reference numerals refer to like elements wherein:

FIG. 1 is a combined perspective view showing a bathroom management apparatus according to a an first embodiment of the present disclosure;

FIG. 2 is an exploded perspective view showing a bathroom management apparatus according to the embodiment;

FIG. 3 is a front perspective view of a frame body illustrated in FIG. 2;

FIG. 4 is a rear perspective view of the frame body illustrated in FIG. 2;

FIG. 5 is a combined perspective view showing a bathroom management apparatus according to another embodiment of the present disclosure;

FIG. 6 is a rear perspective view showing a frame body of a bathroom management apparatus according to another embodiment;

FIG. 7 is a sectional view showing a state in which an upper back bracket illustrated in FIG. 6 is coupled with a mounting bracket;

FIG. 8 is a perspective view showing a state in which an upper back bracket illustrated in FIG. 6 is coupled with a mounting bracket;

FIG. 9 is an enlarged view of a right side frame and a lower frame illustrated in FIG. 6;

FIG. 10 is an enlarged view of a right side frame, an upper frame, and an upper back bracket illustrated in FIG. 6;

FIG. 11 is an enlarged view of a right side frame and a lower back bracket illustrated in FIG. 6;

FIG. 12 is a view showing a bathroom management apparatus according to yet another embodiment of the present disclosure; and

FIG. 13 is a rear perspective view showing a frame body of a bathroom management apparatus according to yet another embodiment.

### DETAILED DESCRIPTION

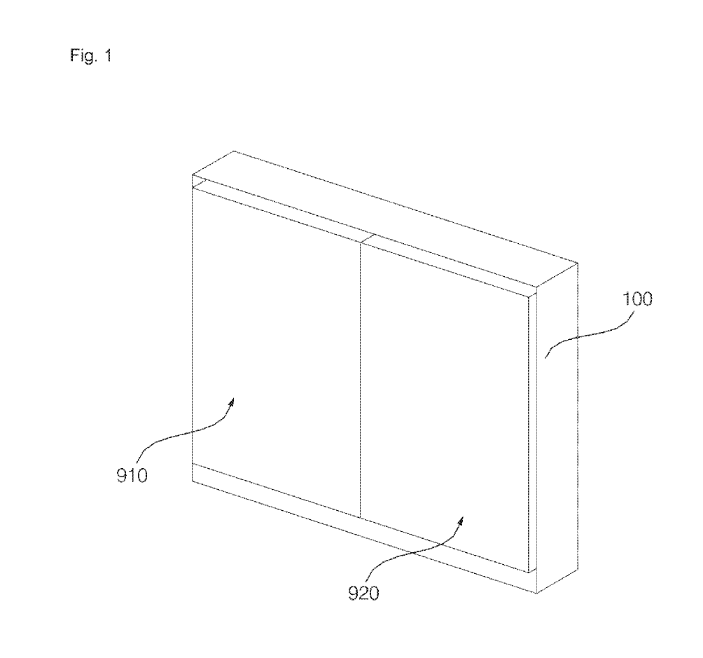

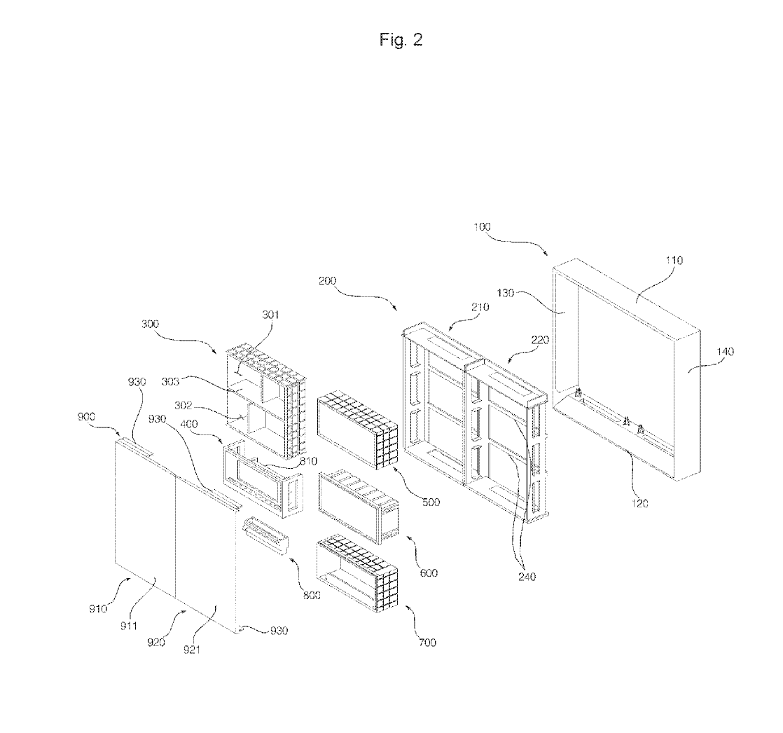

Hereinafter, a bathroom management apparatus according to embodiments of the present disclosure will be described in detail with reference to the accompanying drawings. FIG. 1 is a combined perspective view showing a bathroom management apparatus according to an embodiment of the present disclosure, and FIG. 2 is an exploded perspective view showing a bathroom management apparatus according to the embodiment.

Referring to FIG. 1 and FIG. 2, the bathroom management apparatus, or bathroom cabinet, may include a cabinet 100, a frame 200 installed inside the cabinet 100, a plurality of function modules 300, 400, 500, 600, 700, and 800, and a door 900 configured to open and close the cabinet 100. The cabinet 100 may have a hollow structure and may have a square shape of which a front surface and a rear surface are open. The cabinet 100 may form an upper external appearance, a lower external appearance, a first or left external appearance, and a second or right external appearance.

The cabinet 100 may include an upper panel 110 forming an upper side, a lower panel 120 forming a lower side, a first or left side panel 130 forming a left side, and a second or right side panel 140 forming a right side. The upper panel 110 may connect a top end of the left side panel 130 with a top end of the right side panel 140. The lower panel 120 may connect a bottom end of the left side panel 130 with a bottom end of the right side panel 140.

A left end of the upper panel 110 may be coupled with a top end of the left side panel 130 and a right end of the upper panel 110 may be coupled with a top end of the right side panel 130. Further, a left end of the lower panel 120 may be coupled with a lower end of the left side panel 130, and a right end of the lower panel 120 may be coupled with a bottom end of the right side panel 140.

The frame 200 may include frame bodies 210 and 220 having a square shape corresponding to the cabinet 100 of which a front surface and a rear surface are open, and back brackets 240 provided behind the frame bodies 201 and 220 to be coupled with rear surfaces of the frame bodies 210 and 220. The frame bodies 210 and 220 may reinforce stiffness of the cabinet 100. The bracket 240 may be thicker than the frame bodies 210 and 220 to reinforce the stiffness of the frame bodies 210 and 220.

The frame bodies 210 and 220 may provide a space therein in which the function modules 300, 400, 500, 600, 700, and 800 may be installed. The function modules 300, 400, 500, 600, 700, and 800 may include a towel care module 300, a sterilizing module 400, a secret box module 500, a refrigerating module 600, a charging module 700, and a blower out or exhaust module 800. The towel care module 300, the sterilizing module 400, the secret box module 500, the refrigerating module 600, the charging module 700, and the exhaust module 800 may be independently provided and installed inside the frame bodies 210 and 220 in a module unit. Ribs may protrude from upward, downward, left, and right sides of the function modules 300, 400, 500, 600, 700, and 800, respectively. The ribs protrude from left and right sides of the function modules 300, 400, 500, 600, 700, and 800 may be supported by an inner rib 215 to be described herein protruding in the frame bodies 210 and 220.

The towel care module 300 may include a function division plate 303. The function division plate 303 may vertically divide an inner space of the towel care module 300. The towel care module 300 may include a first storage space 301 for storing towels at a top side of the function division plate 303 and may further include a second storage space 302 for storing the towels at a bottom side of the function division plate 303 and drying and warming the stored towels. The towel care module 300 may include a first independent towel care module having only the first storage space 301 without the function division plate 303 and a second independent towel care module having only the second storage space 302.

The sterilizing module 400 may be used as a toothbrush sterilizer. The sterilizing module 400 may be configured to store toothbrushes, and may include a lamp for irradiating an ultra violet ray to the toothbrushes. The exhaust module 800 may be installed at a bottom side of the sterilizing module 400. An air conditioning module 810 including a blower to suck and blow air to the exhaust module 800 and a heater to heat the air blown from the blower may be installed at a rear portion of the sterilizing module 400 corresponding to a top side of the exhaust module 800 so that the sterilizing module 400 and the air conditioning module 810 may be integrally formed. The exhaust module 800 may exhaust the air blown from the blower into an inside of the bathroom.

The secret box module 500 may be used to store objects to prohibit children and customers from observing or touching the objects. The refrigerating module 600 may be used to refrigerate medicines and cosmetics. The refrigerating module 600 may be installed at a thermoelectric module, which may supply cold air into the refrigerating module 600 and may emit warm air to an outside of the refrigerating module 600.

The charging module 700 may be used to charge electronic devices such as a hair dryer and an electric shaver, for example. The charging module 700 may include a holder to hold the hair dryer and a receptacle to accept a power plug of the electronic device or a power plug of a charger for charging the electronic device.

Two frame bodies 210 and 220 may be provided and may include a first frame body 210 and a second frame body 220 provided adjacent to the first frame body 210. The first frame body 210 and the second frame body 220 may have the same structure. The towel care module 300, the sterilizing module 400, the secret box module 500, the refrigerating module 600, the charging module 700, and the exhaust module 800 may be selected and installed in any combination or duplication according to a need of a consumer. For example, only a plurality of towel care modules 300 may be installed, and two towel care modules 300 and one refrigerating module 600 may be installed in the frame bodies 210 and 220. According to the number of towel care modules 300, the sterilizing modules 400, the secret box modules 500, the refrigerating modules 600, and the charging modules 700 installed in the frame bodies 210 and 220, one or more frame bodies 210 and 220 may be provided.

The door 900 may form a front external appearance of the bathroom management apparatus. The door 900 may open/close an open surface of the cabinet 100. The same number of doors 900 may be provided by the corresponding number of the frame bodies 210 and 220. In an embodiment, since the two frame bodies 210 and 220 are provided, two doors 900 may be provided and may include a first door 910 and a second door 920. The first door 910 may be provided at a forward portion of the first frame body 210 and may open a left side of an open front surface of the cabinet 100, and the second door 920 may be provided at a forward portion of the second frame body 220 and may open a right side of an open front surface of the cabinet 100.

Mirrors 911 and 921 may be provided at front surfaces of the first door 910 and the second door 920, respectively. The mirrors 911 and 921 may be used instead of a mirror inside the bathroom. The mirrors 911 and 921 may include a first mirror 911 provided at a front surface of the first door 910 and a second mirror 921 provided at a front surface of the second door 920.

A hinge 930 may be installed at a rear surface of the door 900. The hinge 930 may include a first hinge member of which one end is coupled with a rear surface of the door and a second hinge member of which one end is rotatably coupled with an opposite end of the first hinge member and an opposite end is rotatably coupled with the frame bodies 210 and 220. The hinges 930 may be installed at a top side and a lower side of a rear surface of the first door 910, respectively, and may be installed at a top side and a bottom side of a rear side of the second door 910.

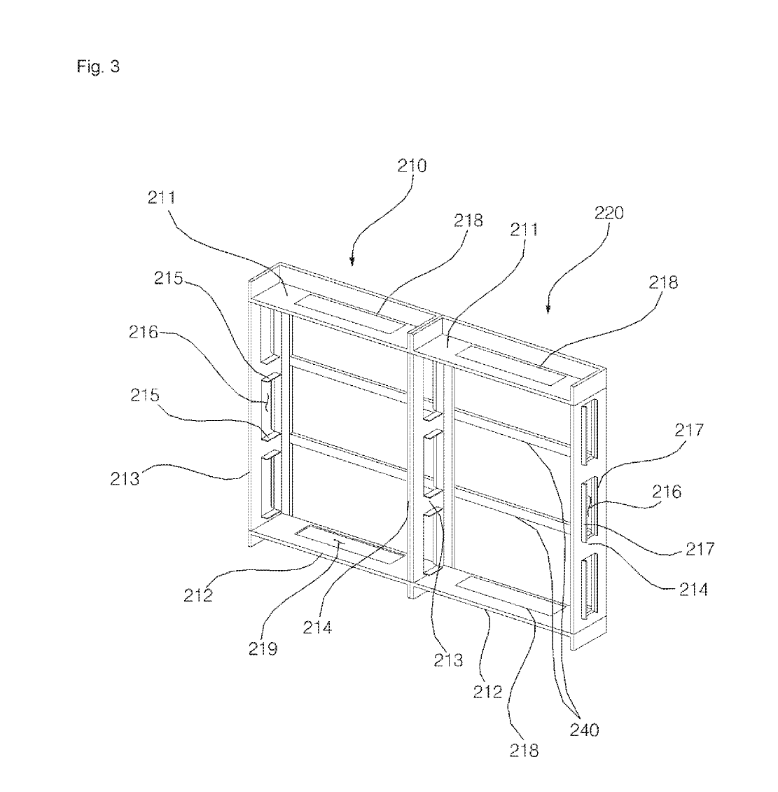

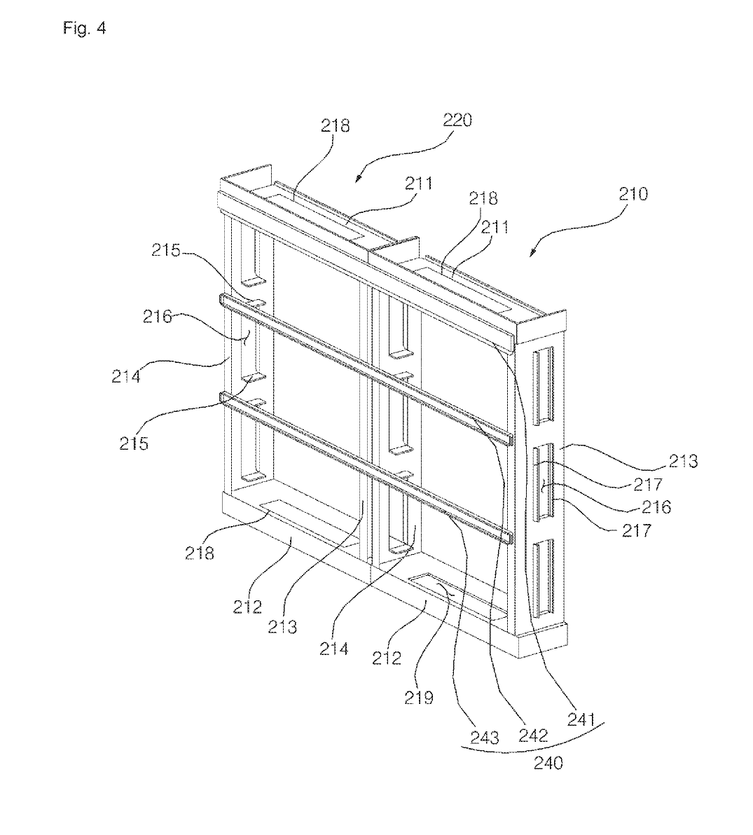

FIG. 3 is a front perspective view of a frame body illustrated in FIG. 2, and FIG. 4 is a rear perspective view of a frame body illustrated in FIG. 2. Referring to FIG. 3 and FIG. 4, each of the first frame body 210 and the second frame body 220 may include an upper frame 211 forming an upper side, a lower frame 212 forming a lower side, a left side frame 213 forming a first or left side, and a right side frame 214 forming a second or right side.

The upper frame 211 may connect a top end of the left side frame 213 with a top end of the right side frame 214. The lower frame 212 may connect a bottom end of the left side frame 213 with a bottom end of the right side frame 214.

A left end of the upper frame 211 may be coupled with a top end of the left side frame 213, and a right end of the upper frame 211 may be coupled with a top end of the right side frame 214. Further, a left end of the lower frame 212 may be coupled with a bottom end of the left side frame 213, and a right end of the lower frame 212 may be coupled with a bottom end of the right side frame 214.

The upper frame 211 and the lower frame 212 may have the same structure. The left side frame 213 and the right side frame 214 may have the same structure. Accordingly, the first frame body 210 and the second frame body 220 may be installed inside the cabinet 100 regardless of upper and lower sides and regardless of left and right sides.

The upper frame 211 may have a shape such that a top surface and a front surface are open and may include a bottom surface, a left surface, a right surface, and a rear surface. Further, the lower frame 212 may have an upside down shape of the upper frame 211 and may have a shape of which a bottom surface and a front surface are open. That is, the lower frame 212 may include a left surface, a right surface, and a rear surface.

First opening portions or slots 216 having a square shape may be formed in the left side frame 213 and the right side frame 214, respectively. The first opening portion 216 may form a path through which wires of the function modules 300, 400, 500, 600, 700, and 800 pass. The same number of the first opening portions 216 may be formed by the corresponding number of the function modules 300, 400, 500, 600, 700, and 800 installed inside the frame bodies 210 and 220.

Inner ribs 215 may be formed at inner sides of the left side frame 213 and the right side frame 214, respectively. The inner ribs 215 may protrude toward the interior of the frame bodies 210 and 220 from a top side and a bottom side of the first opening portion 216.

The inner rib 215 may guide insertion of the function modules 300, 400, 500, 600, 700, and 800 when the function modules 300, 400, 500, 600, 700, and 800 are individually inserted into the frame bodies 210 and 220. After the function modules 300, 400, 500, 600, 700, and 800 are inserted into the frame bodies 210 and 220, the inner rib 215 may support the function modules 300, 400, 500, 600, 700, and 800.

Outer ribs 217 may be formed at outer sides of the left side frame 213 and the right side frame 214, respectively. The outer ribs 217 may protrude to outer sides of the frame bodies 210 and 220 from a front side and a rear side of the first opening portion 216, respectively. The outer ribs 217 may be spaced apart from each other in forward and reward directions while interposing the first opening portion 216 therebetween to form a path through which wires of the function modules 300, 400, 500, 600, 700, and 800 pass after the wires of the function modules 300, 400, 500, 600, 700, and 800 pass through the first opening portion 216.

Cut lines 218 having a square shape may be formed at a bottom surface of the upper frame 211 and a top surface of the lower frame 212, respectively. The cut lines 218 may be formed by partially cutting a bottom surface of the upper frame 211 and a top surface of the lower frame 212 so that a worker may easily separate an inner region divided by the cut lines 218 from a bottom surface of the upper frame 211 an a top surface of the lower frame 212.

When the sterilizing module 400 is installed close to the upper frame, the worker may cut the cut line 218 formed at the upper frame 211 among the cut line 218 formed at the upper frame 211 and the cut line 218 formed at the lower frame 212 to separate an inner region of the upper frame 211 divided by the cut line 218. When the sterilizing module 400 is installed close to the lower frame, the worker may cut the cut line 218 formed at the lower frame 211 among the cut line 218 formed at the upper frame 211 and the cut line 218 formed at the lower frame 212 to separate an inner region of the lower frame 212 divided by the cut line 218.

The second opening portions or slots 219 may be formed at the upper frame 211 and the lower frame 212 when the inner region divided by the cut line 218 is separated by the worker. The exhaust module 800 may be provided between the lower panel 120 of the cabinet 100 and the lower frame 212 of the frame bodies 210 and 220. A top end of the blower out module 800 may be inserted into the second opening portion 219.

The back bracket 240 may be longitudinally formed rightward and leftward. The back bracket 240 may be coupled with the left side frame 213 and the right side frame 214 of the first frame body 210 and the left side frame 213 and the right side frame 214 of the second frame body 220 to connect the first frame body 210 with the second frame body 220. A left end of the back bracket 240 may be coupled with a rear surface of the left side frame 213 of the first frame body 210, and a right end of the back bracket 240 may be coupled with a rear surface of the right side frame 214 of the second frame body 220.

The back bracket 240 may include an upper back bracket 241 located at a highest portion and lower back brackets 242 and 243 provided below the upper back bracket 241. In the present embodiment, the two lower back brackets 242 and 243 may be spaced apart from each other upward and downward.

A length of the back bracket 240 may be determined according to the number of frame bodies 210 and 220. That is, if one of frame bodies 210 and 220 is provided, the back bracket 240 may have a short length. If two or more frame bodies 210 and 220 are provided, the back bracket 240 may have a longer length. The above is described through subsequent embodiments.

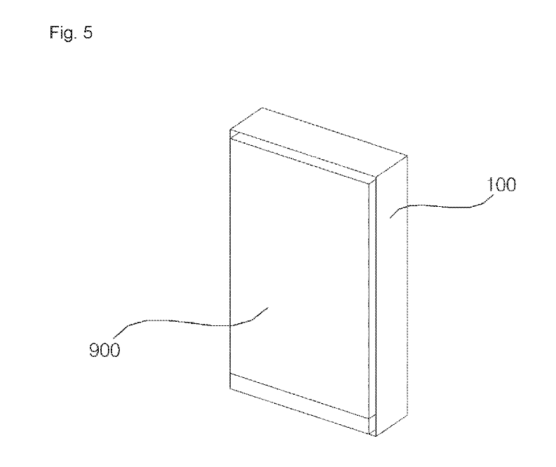

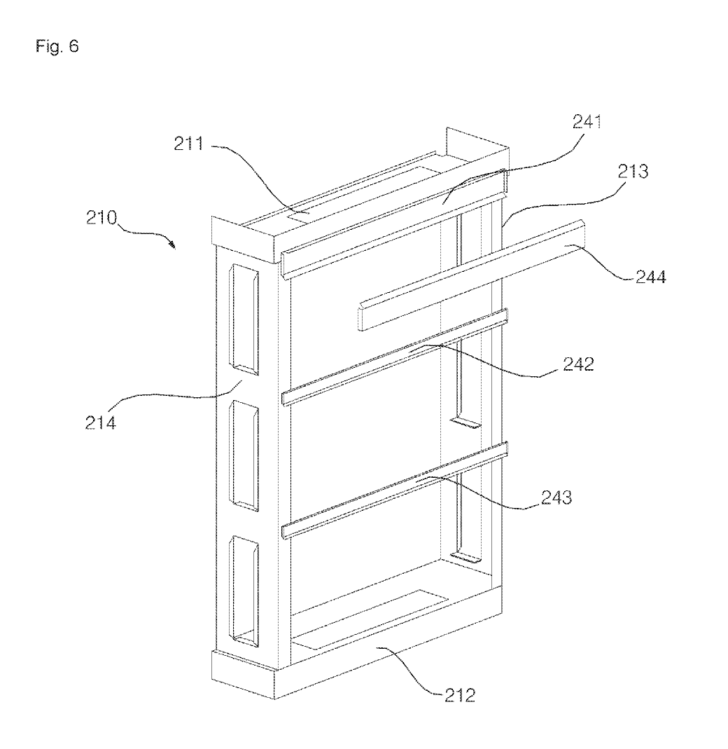

FIG. 5 is a combined perspective view showing a bathroom management apparatus according to another embodiment of the present disclosure, and FIG. 6 is a rear perspective view showing a frame body of a bathroom management apparatus according to another embodiment. Referring to FIG. 5 and FIG. 6, the bathroom management apparatus according to this embodiment differ from the above embodiment. Although two frame bodies 210 and 220 are provided in the above embodiment, one frame body 210 may be provided in this embodiment. Accordingly, a width of the cabinet 100 in embodiment may be narrower than that of the cabinet 100 according to the above embodiment.

Further, lengths of the upper back bracket 241 and lower back brackets 242 and 243 may be shorter than those of the above embodiment. Each left end of the upper back bracket 241 and lower back brackets 242 and 243 may be coupled to a left side frame 213 of the frame body 210, and each right end of the upper back bracket 241 and lower back brackets 242 and 243 may be coupled to a right side panel 214 of the frame body 210. A region between both ends of the upper back bracket 241 may be coupled to an upper frame 211 of the frame body 210.

A mounting bracket 244 for mounting the bathroom management apparatus may be provided at a wall of the bathroom. The mounting bracket 244 may have the same structure as that of the upper back bracket 241 to support the upper back bracket 241. That is, a reversal orientation of the upper back bracket 214 may be used as a mounting bracket 244.

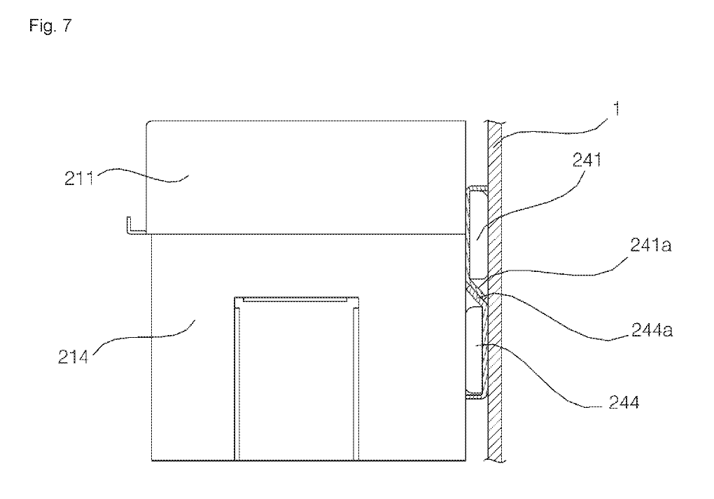

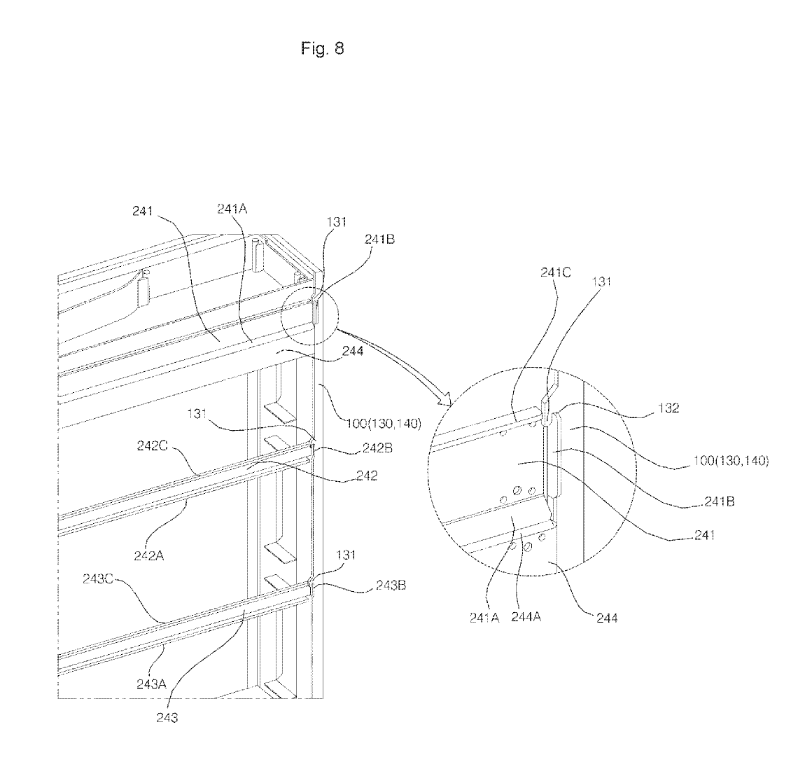

An operation of mounting the bathroom management apparatus on a wall of a bathroom is described with reference to FIG. 7 and FIG. 8 as follows. FIG. 7 is a sectional view showing a state that an upper back bracket illustrated in FIG. 6 is coupled with a mounting bracket, and FIG. 8 is a perspective view showing a state that an upper back bracket illustrated in FIG. 6 is coupled with a mounting bracket.

Referring to FIG. 7 and FIG. 8, a first inclined part (or first inclined tab) 244A may be inclined toward a top end of the mounting bracket 244 to protrude in a forward direction upward. A second inclined part (or second inclined tab) 241A may be inclined toward a bottom end of the upper back bracket 241 to protrude in a rearward direction downward. When the mounting bracket 244 is installed on a bathroom wall 1, the second inclined tab 241A may be inserted into a space between the first inclined tab 244A and the bathroom wall 1 to be supported at the first inclined tab 244A so that the bath management apparatus may be installed on the bathroom wall 1.

Side bending parts or tabs 241B, 242B, and 243B may be formed at both ends of the upper back bracket 241 and the lower back brackets 242 and 243 to be bent rearward. Further, the left side panel 130 and the right side panel 140 of the cabinet 100 may include locking ribs 131 having grooves 132 in which the side bending tabs 241B, 242B, and 243B are inserted, respectively. The locking ribs 131 may protrude to lateral surfaces of the left side panel 130 and the right side panel 140 from a rear surface of the left side panel 130 and the right side panel 140, respectively.

The grooves 132 may be formed at bottom sides of the locking rib 131 so that top sides of the side bending tabs 241B, 242B, and 243B may be inserted into the grooves 132, respectively. The side bending tabs 241B, 242B, and 243B may be inserted into the grooves 132 formed in the locking ribs 131 so that the frame body 210 does not oscillate in rightward and leftward directions when the frame body 210 is installed inside the cabinet 100.

Furthermore, an upper bending part or tab 241C may be formed at a top end of the upper back bracket 241 to protrude rearward. The stiffness of the upper back bracket 241 may be improved due to the upper bending tab 241C, the side bending tab 241B, and the second inclined tab 241A.

Moreover, upper bending parts or tabs 242C and 243C may be formed at upper ends of the lower back brackets 242 and 243 to protrude rearward. Lower bending parts or tabs 242A and 243A may be formed at bottom ends of the lower back brackets 242 and 243 to protrude rearward. The stiffness of the lower back brackets 242 and 243 may be improved due to the lower bending tabs 242C and 243C, the lower bending parts 242A and 243A, and the side bending parts 242B and 243B.

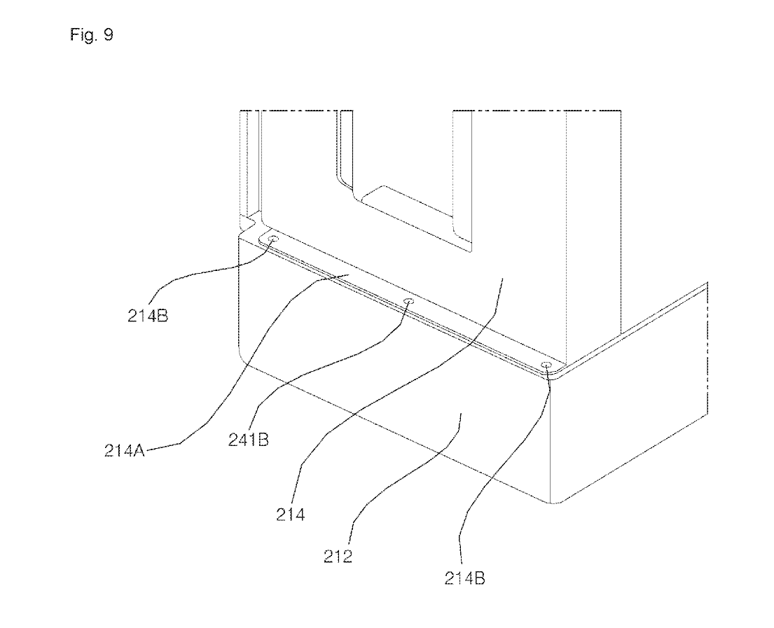

FIG. 9 is an enlarged view of a right side frame and a lower frame illustrated in FIG. 6. Here, a process of manufacturing the frame body 210 is described. Referring to FIG. 6 and FIG. 9, the side frame bending part 214A may be formed at a bottom end of the right side frame 214 to be bent outward. The side frame bending tab 214A may be formed at a top end of the right side frame 214. In addition, since the left side frame 213 has the same structure as that of the right side frame 214, side frame bending tabs 214A may be formed at a top end and a bottom end of the left side frame 213, respectively.

After bottom ends of the left side frame 213 and the right side frame 214 are placed on a top surface of the lower frame 212, the side frame bending tabs 214A formed at a bottom end of the left side frame 213 and a bottom end of the right side frame 214 may be locked with a top surface of the lower frame 212 by a screw so that a bottom end of the left side frame 213 and a bottom end of the right side frame 214 are coupled with the lower frame 212.

Moreover, after a bottom surface of the upper frame 211 is placed on top surfaces of the left side frame 213 and the right side frame 214, the side frame bending tabs 213A and 214A formed at a top end of the left side frame 213 and a top end of the right side frame 214 may be locked with a bottom surface of the upper frame 211 through a screw to lock the top end of the side frame 213 and a top end of the right side frame 214 with the upper frame 211 so that a frame body 210 having a square shape is manufactured. The side frame bending tab 214A may include a plurality of first screw holes 214 through which a screw is formed.

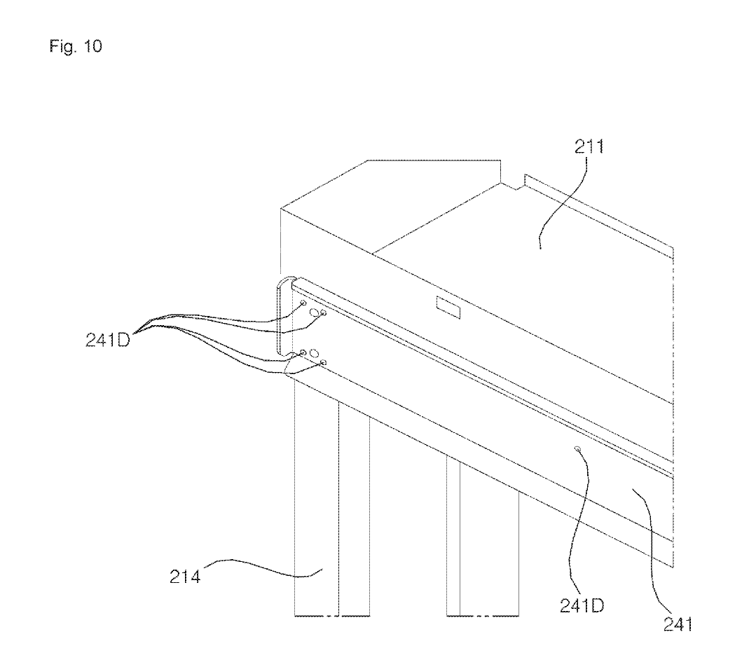

FIG. 10 is an enlarged view of a right side frame, an upper frame, and an upper back bracket illustrated in FIG. 6. Here, an operation of coupling the upper back bracket 241 with the frame body 210 is described. Referring to FIG. 6 and FIG. 10, a left end of the upper back bracket 241 may be coupled with the right side frame 214 and the upper frame 211 with a screw, and a left end of the upper back bracket 241 may be coupled with the left side frame 213 and the upper frame 211 with a screw. Both ends of the upper back bracket 241 and a region between the both ends may be coupled with the upper frame 211 through the screw. A plurality of second locking holes 241D through which the screw may pass may be formed at both ends of the upper back bracket 241 and a region between the both ends. As described above, the upper back bracket 241 may be coupled to the upper frame 211, the left side frame 213, and the right side frame 214 to perform a function of improving coupling force between the upper frame 211, the left side frame 213, and the right side frame 214 as well as a function of installing the mounting bracket 244 on the bathroom wall.

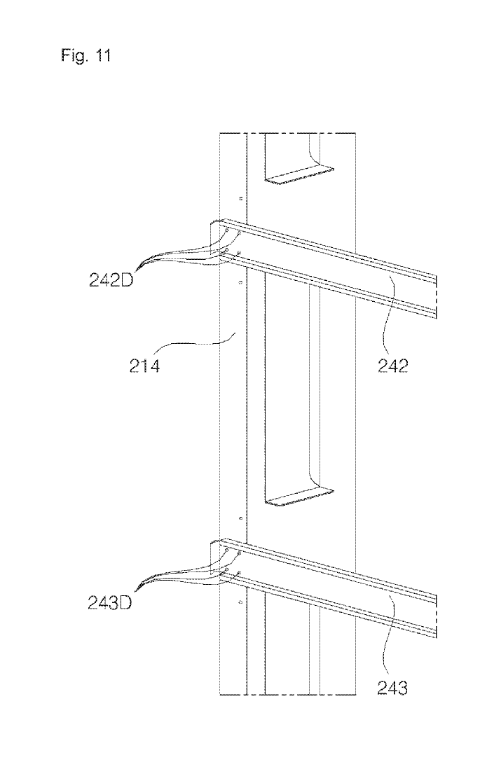

FIG. 11 is an enlarged view of a right side frame and a lower back bracket illustrated in FIG. 6. Here, an operation of coupling the lower back brackets 242 and 243 with the frame body 210 is described. Referring to FIG. 6 and FIG. 11, right ends of the lower back brackets 242 and 243 may be coupled with the right side frame 214 with a screw, and left ends of the lower back brackets 242 and 243 may be coupled with the left side frame 214 with a screw. A plurality of third screw holes 242D and 243D through which the screw passes may be formed at both ends of the lower back brackets 242 and 243.

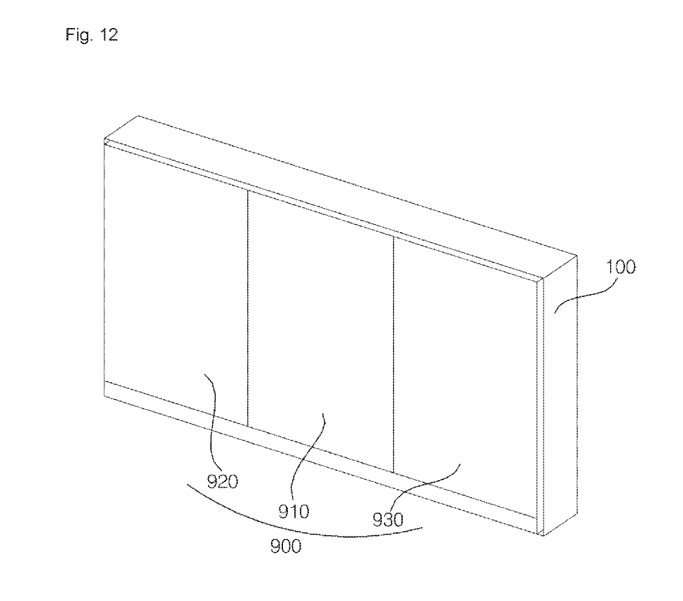

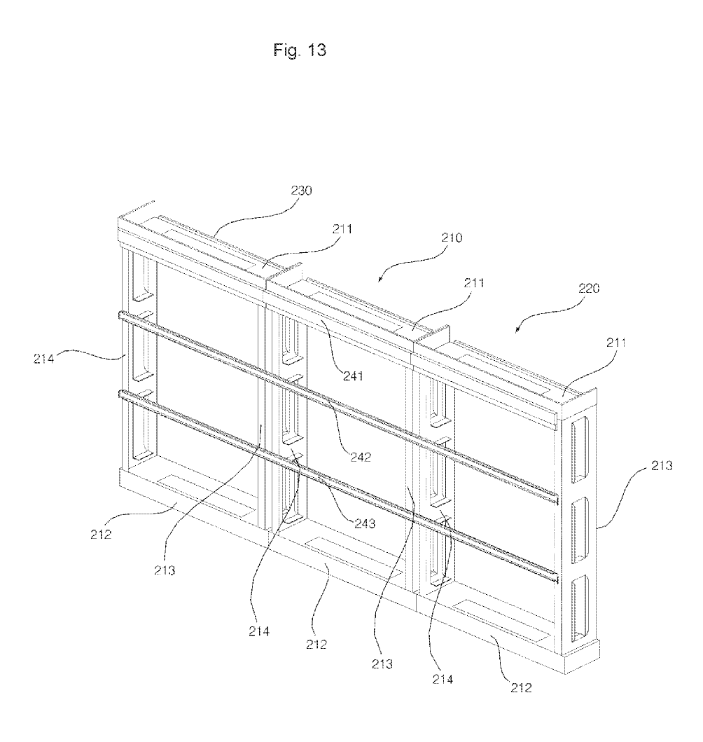

FIG. 12 is a view showing a bathroom management apparatus according to yet another embodiment of the present disclosure, and FIG. 13 is a rear perspective view showing a frame body of a bathroom management apparatus according to yet another embodiment.

Referring to FIG. 12 and FIG. 13, the bathroom management apparatus according to yet another embodiment may differ from the above embodiments. That is, although one or two frame bodies 210 and 220 may be provided, three frame bodies 210, 220, and 230 may be provided in this embodiment, and may include a first frame body 210, a second frame body 220 provided adjacent to the first frame body 210, and a third frame body 230 the first frame body 210 opposite the second frame body 220. Accordingly, a left-right width of the cabinet 100 in this embodiment may be wider than that of the cabinet 100 according to the above embodiments, and may include a first door 910, a second door 920, and a third door 930. The first frame body 210, the second frame body 220, and the third frame body 203 may have similar structures.

Further, lengths of the upper back bracket 241 and the lower back bracket 242 and 243 may be greater than those of the above embodiment. The upper back bracket 241 and the lower back brackets 242 and 243 may be coupled to the left side frames 213 and the right side frames 214 of the first frame body 210, the second frame body 220, and the third frame body 230. Left ends of the upper back bracket 241 and the lower back brackets 242 and 243 may be coupled to the left side frame 213 of the second frame body 220, and right ends of the upper back bracket 241 and the lower back brackets 242 and 243 may be coupled to the right side frame 214 of the third frame body 230. Moreover, both ends of the upper back bracket 241 and a region between the both ends may be coupled to upper frames 211 of the first frame body 210, the second frame body 220, and the third frame body 230, respectively.

As described above, in the bathroom management apparatus according to an embodiment, the number of frame bodies 210, 220, and 230 may be easily extended by connecting a plurality of frame bodies 210, 220, and 230 with each other by using a long back bracket 240. Components mounted on a wall of the bathroom may be used in common by turning the back bracket 240 upside down to use the back bracket 240 as a mounting bracket 244.

Further, a left side frame 213 and a right side frame 214 may have the same structure, and an upper frame 211 and a lower frame 212 may have the same structure so that frame bodies 210, 220, and 230 may be installed regardless of up, down, left, and right sides. Function modules 300, 400, 500, 600, and 700 may be installed in the frame bodies 210, 220, and 230 as an independent module unit to be supported by an inner rib 215 so that it is easy to install the function modules 300, 400, 500, 600, and 700.

A bathroom management apparatus may include a cabinet of which a front surface and a rear surface are open; a frame body installed at an inner side of the cabinet to reinforce stiffness of the cabinet; and a back bracket provided in a rearward direction of the frame body and coupled with both side frames of the frame body, wherein a length of the back bracket is determined according to a number of frame bodies. The bathroom management apparatus may further include a mounting bracket having the same structure as the back bracket and installed on a bathroom wall to support the back bracket.

The frame body may include an upper frame forming an upper side; a lower frame forming a lower side and having the same structure as a structure of the upper frame; first side frame forming a first side; and a second side frame forming a second side and having the same structure as a structure of the first side frame. The bathroom management apparatus may further include a function module including a towel care module, a sterilizing module, a secret box module, a refrigerating module, and a charging module and installed inside the frame body, wherein inner ribs that support the function module are formed at inner sides of the first side frame and the second side frame, respectively.

According to a first objective, the number of frame bodies may be easily extended by connecting a plurality of frame bodies with each other by using a long back bracket. According to a second objective, components mounted on a wall of the bathroom may be used in common by turning the back bracket upside down to use the back bracket as a mounting bracket.

According to a third objective, a first side frame and a second side frame have the same structure, and an upper frame and a lower frame have the same structure so that a frame body may be installed regardless of up, down, left, and right sides. According to a fourth objective, a function module may be installed in the frame body as an independent module unit to be supported by an inner rib so that it is easy to install the function module.

It will be understood that when an element or layer is referred to as being “on” another element or layer, the element or layer can be directly on another element or layer or intervening elements or layers. In contrast, when an element is referred to as being “directly on” another element or layer, there are no intervening elements or layers present. As used herein, the term “and/or” includes any and all combinations of one or more of the associated listed items.

It will be understood that, although the terms first, second, third, etc., may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section could be termed a second element, component, region, layer or section without departing from the teachings of the present invention.

Spatially relative terms, such as “lower”, “upper” and the like, may be used herein for ease of description to describe the relationship of one element or feature to another element(s) or feature(s) as illustrated in the figures. It will be understood that the spatially relative terms are intended to encompass different orientations of the device in use or operation, in addition to the orientation depicted in the figures. For example, if the device in the figures is turned over, elements described as “lower” relative to other elements or features would then be oriented “upper” relative the other elements or features. Thus, the exemplary term “lower” can encompass both an orientation of above and below. The device may be otherwise oriented (rotated 90 degrees or at other orientations) and the spatially relative descriptors used herein interpreted accordingly.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Embodiments of the disclosure are described herein with reference to cross-section illustrations that are schematic illustrations of idealized embodiments (and intermediate structures) of the disclosure. As such, variations from the shapes of the illustrations as a result, for example, of manufacturing techniques and/or tolerances, are to be expected. Thus, embodiments of the disclosure should not be construed as limited to the particular shapes of regions illustrated herein but are to include deviations in shapes that result, for example, from manufacturing.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

Any reference in this specification to “one embodiment,” “an embodiment,” “example embodiment,” etc., means that a particular feature, structure, or characteristic described in connection with the embodiment is included in at least one embodiment of the invention. The appearances of such phrases in various places in the specification are not necessarily all referring to the same embodiment. Further, when a particular feature, structure, or characteristic is described in connection with any embodiment, it is submitted that it is within the purview of one skilled in the art to effect such feature, structure, or characteristic in connection with other ones of the embodiments.

Although embodiments have been described with reference to a number of illustrative embodiments thereof, it should be understood that numerous other modifications and embodiments can be devised by those skilled in the art that will fall within the spirit and scope of the principles of this disclosure. More particularly, various variations and modifications are possible in the component parts and/or arrangements of the subject combination arrangement within the scope of the disclosure, the drawings and the appended claims. In addition to variations and modifications in the component parts and/or arrangements, alternative uses will also be apparent to those skilled in the art.

What is claimed is:

1. A bathroom management apparatus comprising:
a cabinet having a front opening and a rear opening;
at least one frame body configured to be attached to an inner side of the cabinet to reinforce a stiffness of the cabinet; and
at least one back bracket attached to a rear of the at least one frame body and coupled to a first side frame and a second side frame of the frame body, wherein a length of the at least one back bracket is determined according to a number of frame bodies provided.

2. The bathroom management apparatus of claim 1, further including a mounting bracket having the same shape as the at least one back bracket and installed on a bathroom wall to support the at least one back bracket.

3. The bathroom management apparatus of claim 2, wherein the at least one back bracket includes:
an upper back bracket; and
at least one lower back bracket arranged below the upper back bracket, wherein the mounting bracket includes a first inclined tab inclined from the bathroom wall toward a top end of the mounting bracket to protrude upward in a forward direction, and the upper back bracket includes a second inclined tab corresponding to the first inclined tab and inclined toward a bottom end of the upper back bracket from the at least one frame body to protrude downward in a rearward direction.

4. The bathroom management apparatus of claim 1, wherein the at least one back bracket includes side bending tabs formed at both ends of the at least one back bracket and extending rearward from the at least one frame body, and the cabinet includes locking ribs having grooves in which the side bending tabs are inserted, respectively.

5. The bathroom management apparatus of claim 4, wherein the cabinet includes an upper panel, a lower panel, a first side panel forming a left side, and a second side panel forming a right side, and wherein the locking ribs are formed at the first side panel and the second side panel, respectively.

6. The bathroom management apparatus of claim 1, wherein the at least one frame body includes:
an upper frame;
a lower frame having the same shape as the upper frame;
a first side frame forming a left side; and
a second side frame forming a right side and having the same shape as the first side frame.

7. The bathroom management apparatus of claim 6, further including:
at least one function module installed inside the at least one frame body, wherein the at least one function module is one of a towel care module, a sterilizing module, a secret box module, a refrigerating module, and a charging module, and wherein the first side frame and the second side frame each include inner ribs that support the at least one function module and are formed at inner sides of the first side frame and the second side frame, respectively.

8. The bathroom management apparatus of claim 7, wherein first slots are formed at the first side frame and the second side frame, respectively, and the inner rib protrudes toward an inner side of the first slot from a top side and a bottom side of the first slot.

9. The bathroom management apparatus of claim 8, wherein the first and second side frames each further include outer ribs that protrude to an outer side of the first slot from a front side and a rear side of the first slot, respectively.

10. The bathroom management apparatus of claim 6, wherein the at least one back bracket includes:
an upper back bracket coupled to the first side frame, the second side frame, and the upper frame; and
at least one lower back bracket arranged below the upper back bracket and configured to be coupled with the first side frame and the second side frame.

11. The bathroom management apparatus of claim 6, wherein cut lines are formed at the upper frame and the lower frame and are configured to be cut to separate an inner region of the upper frame divided by the cut lines so that the upper frame and the lower frame form a second slot.

12. The bathroom management apparatus of claim 1, wherein the at least one frame body includes a first frame body and a second frame body having the same structure as the first frame body and provided adjacent to the first frame body, and the at least one back bracket is coupled to side frames of both the first frame body and the second frame body to connect the first frame body to the second frame body.

13. The bathroom management apparatus of claim 1, wherein the at least one frame body includes a first frame body, a second body having the same structure as the first frame body and provided adjacent to the first frame body, and a third frame body having the same structure as the first frame body and provided adjacent to the first frame body opposite the second frame body, and the at least one back bracket is coupled to side frames of the first frame body, the second frame body, and the third frame body to connect the first frame body, the second frame body, and the third frame body to each other.
